(19) United States
(12) Patent Application Publication
(10) Pub. No.: US 2005/0143789 A1
(43) Pub. Date: Jun. 30, 2005

(54) Methods and systems for stimulating a peripheral nerve to treat chronic pain

(76) Inventors: Todd K. Whitehurst, Santa Clarita, CA (US); Kristen N. Jaax, Saugus, CA (US)

Correspondence Address: STEVEN L. NICHOLS;RADER, FISHMAN & GRAVER PLLC, 10653 S. RIVER FRONT PARKWAY, SUITE 150, SOUTH JORDAN, UT, 84095, US

(21) Appl. No.: 11/067,111

(22) Filed: Feb. 25, 2005

Related U.S. Application Data
(63) Continuation-in-part of application No. 10/057,116, filed on Jan. 24, 2002.
(60) Provisional application No. 60/265,009, filed on Jan. 30, 2001.

(51) Int. Cl. A61N 1/18

(52) U.S. Cl. 607/46

(57) ABSTRACT

Methods of treating chronic pain within a patient include applying at least one stimulus to a peripheral nerve within the patient with an implanted system control unit in accordance with one or more stimulation parameters. The stimulus is configured to treat the chronic pain. Systems for treating chronic pain within a patient include a system control unit configured to apply a stimulus to a peripheral nerve within the patient in accordance with one or more stimulation parameters. The system control unit is implanted within the patient and the stimulus is configured to treat the chronic pain.

### RELATED APPLICATIONS

The present application is a continuation-in-part application of U.S. application Ser. No. 10/057,116, filed Jan. 24, 2002, which application claims the benefit of Provisional Application Ser. No. 60/265,009, filed Jan. 30, 2001. Both applications are incorporated herein by reference in their entireties.

### BACKGROUND

Neuropathic pain is the result of a malfunction somewhere in the nervous system. The site of the nervous system injury or malfunction can be either in the peripheral or central nervous system. Neuropathic pain is often triggered by a disease or an injury. However, many diseases and injuries often do not clearly involve the nervous system and yet, the resulting pain may persist for months or years beyond the apparent healing of any damaged tissues. The pain is frequently described as having a burning, lancinating, or electric shock characteristic. Persistent allodynia—pain resulting from a non-painful stimulus, such as light touch—is also a common characteristic of neuropathic pain. Neuropathic pain is frequently chronic.

Chronic pain is a major public health problem. It is estimated that chronic pain affects fifteen to thirty-three percent of the United States population or as many as seventy million people. Chronic pain disables more people than cancer or heart disease and costs Americans more than both diseases combined. Chronic pain costs an estimated seventy billion dollars a year in medical costs, lost working days, and workers' compensation.

Patients with chronic neuropathic pain currently have very few treatment alternatives. Chronic pain is often poorly controlled by medication. Surgery is often ineffective, as the pain may persist even after surgery. Chronic pain may also be controlled through the use of a transcutaneous electrical nerve stimulation (TENS) system which masks local pain sensations with a fine tingling sensation. However, TENS devices can produce significant discomfort and can only be used intermittently.

### SUMMARY

Methods of treating chronic pain within a patient include applying at least one stimulus to a peripheral nerve within the patient with an implanted system control unit in accordance with one or more stimulation parameters. The stimulus is configured to treat the chronic pain.

Systems for treating chronic pain within a patient include a system control unit configured to apply a stimulus to a peripheral nerve within the patient in accordance with one or more stimulation parameters. The system control unit is implanted within the patient and the stimulus is configured to treat the chronic pain.

### BRIEF DESCRIPTION OF THE DRAWINGS

The accompanying drawings illustrate various embodiments of the present invention and are a part of the specification. The illustrated embodiments are merely examples of the present invention and do not limit the scope of the invention.

FIG. 1 is a diagram of the human nervous system according to principles described herein.

FIG. 2 illustrates an exemplary system control unit (SCU) that may be implanted within a patient and used to apply electrical stimulation to a peripheral nerve and/or infuse one or more drugs into the peripheral nerve to treat chronic pain according to principles described herein.

FIG. 3 illustrates an exemplary BION microstimulator that may be used as the SCU according to principles described herein.

FIG. 4 illustrates leadless microstimulator subcutaneously implanted within a patient in a location where the patient feels pain according to principles described herein.

FIG. 5 shows that one or more catheters may be coupled to the microstimulator according to principles described herein.

FIG. 6 depicts a number of SCUs configured to communicate with each other and/or with one or more external devices according to principles described herein.

Throughout the drawings, identical reference numbers designate similar, but not necessarily identical, elements.

### DETAILED DESCRIPTION

Methods and systems for treating chronic pain within a patient are described herein. A system control unit (SCU) is implanted within the patient. The SCU is configured to apply at least one stimulus to a peripheral nerve within the patient in accordance with one or more stimulation parameters. The stimulus is configured to treat the chronic pain and may include electrical stimulation, drug stimulation, or both. Consequently, as used herein and in the appended claims, the term “stimulus” will broadly refer to either an electrical stimulation, a drug therapy or stimulation, or both.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

The terms “chronic pain” and “chronic neuropathic pain” will be used herein and in the appended claims, unless otherwise specifically denoted, to refer to any type of pain that is the result of a malfunction somewhere in the nervous system. Chronic pain has a variety of causes and associated conditions. For example, some types of chronic pain are caused by diseases such as, but not limited to, diabetes, uremia, AIDs, or nutritional deficiencies. Various vascular or collagen disorders such as atherosclerosis, systemic lupus erythematosus, scleroderma, sarcoidosis, rheumatoid arthritis, and polyarteritis nodosa may also cause chronic pain. Other causes of chronic pain include, but are not limited to, compression or entrapment, direct trauma, penetrating injuries, contusions, fractured or dislocated bones, tumors, intraneural hemorrhages, exposure to cold or radiation, prolonged use of crutches, staying in one position for too long, and any other type of pressure involving any peripheral nerve such as the ulnar, radial, or peroneal nerves.

One type of chronic pain is known as painful peripheral neuropathy. Painful peripheral neuropathy includes the conditions known as diabetic neuropathy and traumatic peripheral nerve injury. Painful peripheral neuropathy is typically characterized by a numbness or tingling in the toes which slowly spreads upward. Occasionally, the condition affects the front of the thighs or starts in the fingers and moves up the hands. At times, symptoms may be barely noticeable, and at other times, especially at night, they may be almost unbearable. For some, symptoms are constant. Common symptoms include tingling, prickling, numbness, burning or freezing pain, sharp pain, extreme sensitivity to touch, muscle weakness, and/or loss of balance and/or coordination. Painful peripheral neuropathy affects more than two million Americans, most of whom are older adults.

Another type of chronic pain is known as Post-Herpetic Neuralgia (PHN). Herpes zoster, or “shingles”, is an infection caused by the varicella-zoster virus, which is the virus that causes chickenpox. Shingles occurs in people who have had chickenpox and represents a reactivation of the dormant varicella-zoster virus. Shingles typically causes a localized rash and associated pain, which usually subside within three to five weeks. However, sometimes the pain will continue long after the rash has cleared. This persistent pain is known as postherpetic neuralgia.

Postherpetic neuralgia is the most common complication of shingles. It affects approximately half of the people over age sixty who develop shingles and approximately seventy-five percent of the people over age seventy who develop shingles. Postherpetic neuralgia results from damage to nerve fibers that occurs when a person has shingles. Common symptoms of post-herpetic neuralgia include sharp and jabbing pain, a burning sensation, deep and aching pain, extreme skin sensitivity, itching, numbness, muscle weakness, muscle tremor, and paralysis.

Reflex Sympathetic Dystrophy (RSD), also known as Complex Regional Pain Syndrome, is another type of chronic pain. The primary clinical feature of RSD is pain in one or more extremities that is severe, constant, burning, deep, and/or aching. All tactile stimulation of the skin (e.g., wearing clothing or a light breeze) may be painful. Other symptoms of RSD include shiny, dry, or scaly skin and swelling.

Fibromyalgia Syndrome (FMS) is yet another type of chronic pain. FMS is a disorder of unknown etiology affecting an estimated two to four percent of the general population. Women tend to suffer from FMS more often than men. Symptoms of FMS include constant aching all over the body, fatigue, morning stiffness, sleep disturbance, paresthesias, and headaches.

Another type of chronic pain, failed back surgery syndrome (FBSS), refers to patients who have undergone one or more surgical procedures and continue to experience pain. Included in this condition are recurring disc herniation, epidural scarring, and injured nerve roots.

Arachnoiditis, a disease that occurs when the membrane in direct contact with the spinal fluid becomes inflamed, causes chronic pain by pressing on the nerves. It is unclear what causes this condition.

Chronic pelvic pain syndrome may present patients with multiple overlapping symptoms that frequently involve pelvic pain, urgency/frequency syndrome, urge incontinence, fecal incontinence, functional bowel disease, dyspareunia, vestibulitis, and/or dysfunctional voiding. Neurostimulation of the sacral nerve roots has been demonstrated to relieve pelvic pain in patients with intractable chronic pelvic pain.

Additional forms of chronic peripheral pain include, but are not limited to occipital neuralgia, certain types of cardiac pain, and certain types of back pain. Many of these patients are treated symptomatically for their pain.

The above-mentioned diseases and disorders are merely exemplary of the many different types of chronic pain that may be treated via electrical stimulation and/or drug stimulation of one or more peripheral nerves. As will be described in more detail below, a system control unit (SCU), such as a microstimulator, configured to apply at least one stimulus to one or more peripheral nerves may be implanted within a patient to treat chronic pain.

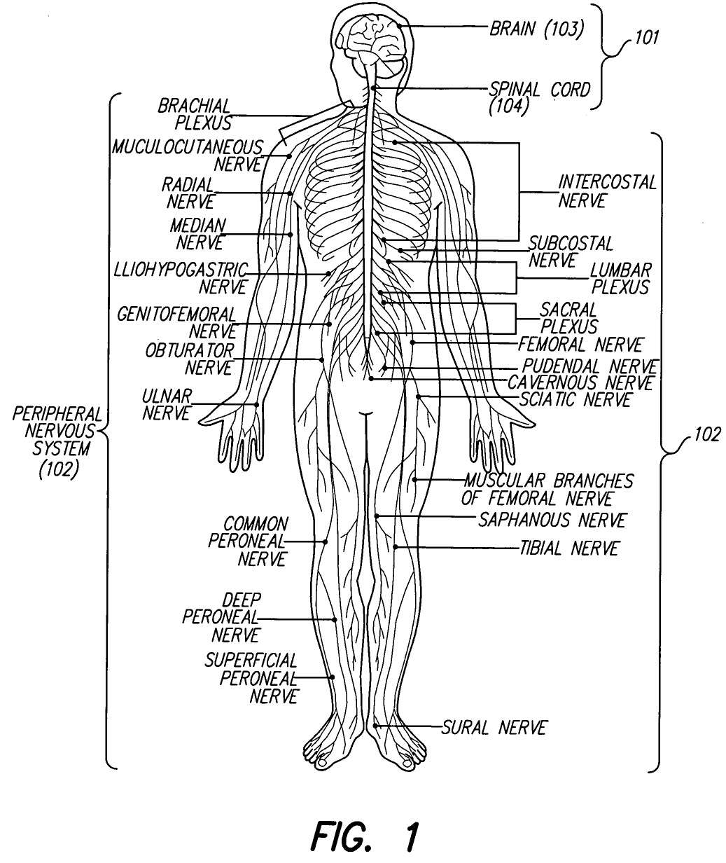

FIG. 1 is a diagram of the human nervous system. The nervous system may be divided into a central nervous system (101) and a peripheral nervous system (102). The central nervous system (101) includes the brain (103) and the spinal cord (104). The peripheral nervous system (102) includes a number of nerves that branch from various regions of the spinal cord (104). For example, the peripheral nervous system (102) includes, but is not limited to, the brachial plexus, the musculocutaneous nerve, the radial nerve, the median nerve, the lliohypogastric nerve, the genitorfemoral nerve, the obturator nerve, the ulnar nerve, the peroneal nerve, the sural nerve, the tibialis nerve, the saphenous nerve, the femoral nerve, the sciatic nerve, the cavernous nerve, the pudendal nerve, the sacral plexus, the lumbar plexus, the subcostal nerve, the occipital nerves (including, but not limited to, the greater occipital nerve, the lesser occipital nerve, and the third occipital nerve), and the intercostal nerves. Each of these peripheral nerves provides innervation to and from different parts of the body.

Many of the above mentioned peripheral nerves, especially those in the extremities and in the thorax, lie relatively close to the surface of the skin and are surrounded by relatively few if any surgical barriers. Thus, as will be described in more detail below, an SCU may be implanted subcutaneously near a peripheral nerve via injection and/or endoscopic means and used to treat chronic pain. A more complicated surgical procedure may be used to access a particular peripheral nerve that is more deeply embedded within a patient or surrounded by a surgical barrier, such as scar tissue.

In some embodiments, at least one stimulus is applied to a peripheral nerve within a patient to treat chronic pain. The peripheral nerve may be any peripheral nerve within the patient, including, but not limited to, the peripheral nerves listed in connection with FIG. 1. For instance, the stimulus may be applied to an occipital nerve to treat occipital neuralgia. As used herein and in the appended claims, the term “occipital nerve” will be used to refer to any of the occipital nerves in the peripheral nervous system including, but not limited to, the greater occipital nerve, the lesser occipital nerve, and the third occipital nerve. The stimulus may be applied to the peripheral nerve directly. Alternatively and/or additionally, the stimulus may be applied to an organ or other tissue located in an area where a patient feels pain so that the peripheral nerves in that area are stimulated. Hence, it will be recognized that “stimulating a peripheral nerve” refers to applying a stimulus to the peripheral nerve directly and/or applying a stimulus to an organ or other tissue located relatively near the peripheral nerve.

The stimulus applied to the peripheral nerve may include electrical stimulation, also known as neuromodulation. Electrical stimulation will be described in more detail below. The stimulus may additionally or alternatively include drug stimulation. As will be described in more detail below, therapeutic dosages of one or more drugs may be infused into a peripheral nerve or into a site near the peripheral nerve to treat chronic pain.

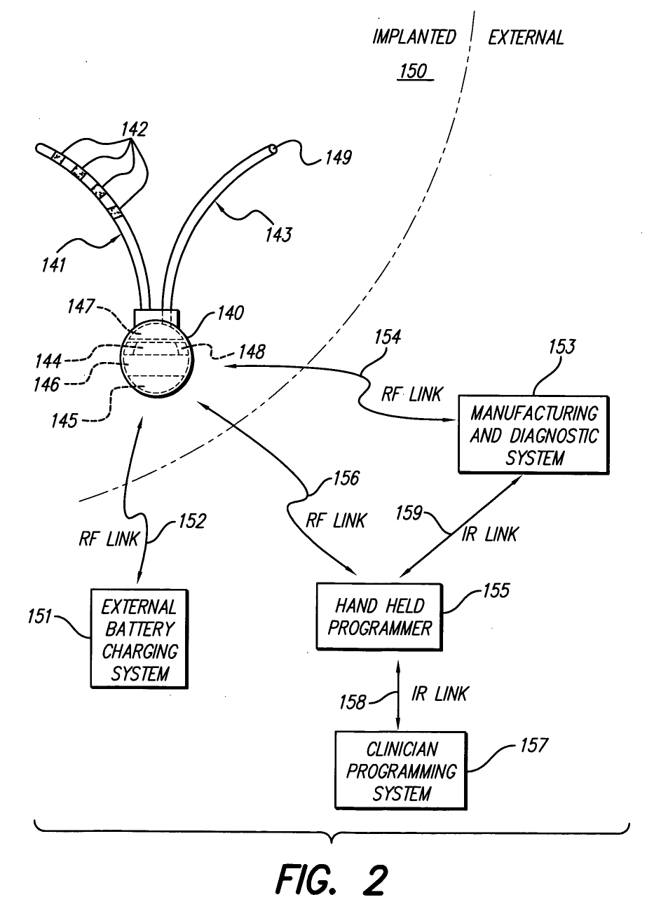

In some embodiments, the electrical stimulation and/or the drug infusion may be performed by one or more implantable system control units (SCUs). FIG. 2 illustrates an exemplary SCU (140) that may be implanted within a patient (150) and used to apply a stimulus to a peripheral nerve to treat chronic pain, e.g., an electrical stimulation to a peripheral nerve, an infusion of one or more drugs into the peripheral nerve, or both.

FIG. 2 shows a lead (141) having a proximal end that may be coupled to the SCU (140) and that may include a number of electrodes (142) configured to apply a stimulation current to a peripheral nerve. In some embodiments, the lead (141) includes anywhere between two and sixteen electrodes (142). However, the lead (141) may include any number of electrodes (142) as best serves a particular application. The electrodes (142) may be arranged as an array, for example, having at least two or at least four collinear electrodes. In some embodiments, the electrodes are alternatively inductively coupled to the SCU (140). The lead (141) may be thin (e.g., less than 3 millimeters in diameter) such that the lead (141) may be positioned near a nerve axon, for example. Alternatively, as will be described in more detail below, the SCU (140) may be leadless.

As illustrated in FIG. 2, the SCU (140) may include a number of components. A power source (145) is configured to output voltage used to supply the various components within the SCU (140) with power. The power source (145) may be a primary battery, a rechargeable battery, a capacitor, or any other suitable power source. A coil (148) is configured to receive and/or emit a magnetic field (also referred to as a radio frequency (RF) field) that is used to communicate with or receive power from one or more external devices (151, 153, 155). Such communication and/or power transfer may include, but is not limited to, transcutaneously receiving data from the external device, transmitting data to the external device, and/or receiving power used to recharge the power source (145).

For example, an external battery charging system (EBCS) (151) may provide power used to recharge the power source (145) via an RF link (152). External devices including, but not limited to, a hand held programmer (HHP) (155), clinician programming system (CPS) (157), and/or a manufacturing and diagnostic system (MDS) (153) may be configured to activate, deactivate, program, and test the SCU (140) via one or more RF links (154, 156). One or more of these external devices (153, 155, 157) may also be used to control the infusion of one or more drugs into a peripheral nerve to treat chronic pain.

Additionally, if multiple external devices are used in the treatment of a patient, there may be some communication among those external devices, as well as with the implanted SCU (140). For example, the CPS (157) may communicate with the HHP (155) via an infrared (IR) link (158) or via any other suitable communication link. Likewise, the MDS (153) may communicate with the HHP (155) via an IR link (159) or via any other suitable communication link.

The HHP (155), MDS (153), CPS (157), and EBCS (151) are merely illustrative of the many different external devices that may be used in connection with the SCU (140). Furthermore, it will be recognized that the functions performed by the HHP (155), MDS (153), CPS (157), and EBCS (151) may be performed by a single external device. One or more of the external devices (153, 155, 157) may be embedded in a seat cushion, mattress cover, pillow, garment, belt, strap, pouch, or the like.

The SCU (140) may also include electrical circuitry (144) configured to produce electrical stimulation pulses that are delivered to the peripheral nerve via the electrodes (142). In some embodiments, the SCU (140) may be configured to produce monopolar stimulation. The SCU (140) may alternatively or additionally be configured to produce bipolar stimulation. Monopolar electrical stimulation is achieved, for example, using the stimulator case as an indifferent electrode. Bipolar electrical stimulation is achieved, for example, using one of the electrodes of the electrode array as an indifferent electrode. The electrical circuitry (144) may include one or more processors configured to decode stimulation parameters and generate the stimulation pulses. In some embodiments, the SCU (140) has at least four channels and drives up to sixteen electrodes or more. The electrical circuitry (144) may include additional circuitry such as capacitors, integrated circuits, resistors, coils, and the like configured to perform a variety of functions as best serves a particular application.

The SCU (140) may also include a programmable memory unit (146) for storing one or more sets of data and/or stimulation parameters. The stimulation parameters may include, but are not limited to, electrical stimulation parameters and drug stimulation parameters. The programmable memory (146) allows a patient, clinician, or other user of the SCU (140) to adjust the stimulation parameters such that the electrical stimulation and/or drug stimulation are at levels that are safe and efficacious for a particular type of chronic pain and/or for a particular patient. Electrical stimulation and drug stimulation parameters may be controlled independently. However, in some instances, the electrical stimulation and drug stimulation parameters are coupled, e.g., electrical stimulation may be programmed to occur only during drug stimulation. The programmable memory (146) may be any type of memory unit such as, but not limited to, random access memory (RAM), static RAM (SRAM), a hard drive, or the like.

The electrical stimulation parameters may control various parameters of the stimulation current applied to a peripheral nerve including, but not limited to, the frequency, pulse width, amplitude, burst pattern (e.g., burst on time and burst off time), duty cycle or burst repeat interval, ramp on time and ramp off time of the stimulation current that is applied to the peripheral nerve. The drug stimulation parameters may control various parameters including, but not limited to, the amount of drugs infused into the peripheral nerve, the rate of drug infusion, and the frequency of drug infusion.

Specific electrical stimulation and drug stimulation parameters may have different effects on different types of chronic pain. Thus, in some embodiments, the electrical stimulation and/or drug stimulation parameters may be adjusted by the patient, a clinician, or other user of the SCU (140) as best serves a particular type of chronic pain. The electrical stimulation and/or drug stimulation parameters may also be automatically adjusted by the SCU (140), as will be described below. For example, the amplitude of the stimulus current applied to a peripheral nerve may be adjusted to have a relatively low value to target relatively large diameter fibers of a peripheral nerve. The SCU (140) may also increase excitement of a peripheral nerve by applying a stimulation current having a relatively low frequency to the peripheral nerve (e.g., less than 100 Hz). The SCU (140) may also decrease excitement of a peripheral nerve by applying a relatively high frequency to the peripheral nerve (e.g., greater than 100 Hz). The SCU (140) may also be programmed to apply the stimulation current to a peripheral nerve intermittently or continuously.

As shown in FIG. 2, a pump (147) may also be included within the SCU (140). The pump (147) is configured to store and dispense one or more drugs through a catheter (143). The catheter (143) is coupled at a proximal end to the SCU (140) and may have an infusion outlet (149) for infusing dosages of the one or more drugs into a predetermined site within a peripheral nerve. In some embodiments, the SCU (140) may include multiple catheters (143) and/or pumps (147) for storing and infusing dosages of the one or more drugs into predetermined sites within the peripheral nerve.

The SCU (140) of FIG. 2 may be implanted within the patient (150) using any suitable surgical procedure such as, but not limited to, injection, small incision, open placement, laparoscopy, or endoscopy. In some instances, the SCU (140) may be implanted at a site that is any distance from a target peripheral nerve with the lead (141) and/or the catheter (143) being routed to the target peripheral nerve. The SCU (140) itself may also or alternatively be implanted at or near the peripheral nerve.

The SCU (140) of FIG. 2 may alternatively be subcutaneously implanted just beneath the skin of the patient (150) in a location where the patient (150) feels pain. This location may be a considerable distance from the peripheral nerve causing the pain. For example, the peripheral nerve causing the pain may be located deep within the body of the patient (150) or in a location that is otherwise removed from the location where the patient (150) feels the pain. In these instances, the SCU (140) may be configured to stimulate neural tissue in the local vicinity of the implanted SCU (140). Stimulation of the neural tissue in the local vicinity of the implanted SCU (140) may effectively stimulate the peripheral nerve causing the pain because the neural tissue close to the surface of the skin is connected to the peripheral nerve via a number of small nerve fibers. Thus, stimulation of the neural tissue in the local vicinity of the implanted SCU (140) is advantageous in many instances where the peripheral nerve causing the pain is located deep within the body of the patient (150) or in a location that is otherwise removed from the location where the patient (150) feels pain and where invasive surgical procedures are required to implant the SCU (140), lead (141), and/or catheter (143) deep within the patient (150).

The SCU (140) of FIG. 2 is illustrative of the many types of SCUs that may be used to apply electrical stimulation to a peripheral nerve and/or infuse one or more drugs into the peripheral nerve to treat chronic pain. For example, the SCU (140) may include an implantable pulse generator (IPG) coupled to one or more leads (141) having a number of electrodes (142). In the case of drug stimulation only, the SCU (140) comprises a pump. Alternatively, the SCU (140) may be an implantable microstimulator, such as a BION® microstimulator (Advanced Bionics® Corporation, Valencia, Calif.). The following listed patents describe various details associated with the manufacture, operation, and use of BION implantable microstimulators, and are all incorporated herein by reference in their respective entireties:

| Application/Patent/ | Filing/Publication | |
|---|---|---|
| Publication No. | Date | Title |
| U.S. Pat. No. 5,193,539 | Issued Mar 16, 1993 | Implantable |
| | | Microstimulator |
| U.S. Pat. No. 5,193,540 | Issued Mar 16, 1993 | Structure and Method |
| | | of Manufacture of an |
| | | Implantable |
| | | Microstimulator |
| U.S. Pat. No. 5,312,439 | Issued May 17, 1994 | Implantable Device |
| | | Having an |
| | | Electrolytic Storage |
| | | Electrode |
| U.S. Pat. No. 6,185,452 | Issued Feb. 6, 2001 | Battery-Powered |
| | | Patient Implantable |
| | | Device |
| U.S. Pat. No. 6,164,284 | Issued Dec. 26, 2000 | System of Implantable |
| | | Devices For |
| | | Monitoring and/or |
| | | Affecting Body |
| | | Parameters |
| U.S. Pat. No. 6,208,894 | Issued Mar. 27, 2001 | System of Implantable |
| | | Devices For |
| | | Monitoring and/or |
| | | Affecting Body |
| | | Parameters |
| U.S. Pat. No. 6,051,017 | Issued Apr. 18, 2000 | Implantable |
| | | Microstimulator and |
| | | Systems Employing |
| | | Same |

The pump or controlled drug release device described herein may include any of a variety of different drug delivery systems. Controlled drug release devices based upon a mechanical or electromechanical infusion pump may be used. In other examples, the controlled drug release device can include a diffusion-based delivery system, e.g., erosion-based delivery systems (e.g., polymer-impregnated with drug placed within a drug-impermeable reservoir in communication with the drug delivery conduit of a catheter), electrodiffusion systems, and the like. Another example is a convective drug delivery system, e.g., systems based upon electroosmosis, vapor pressure pumps, electrolytic pumps, effervescent pumps, piezoelectric pumps and osmotic pumps.

Exemplary controlled drug release devices suitable for use as described herein include, but are not necessarily limited to, those disclosed in U.S. Pat. Nos. 3,760,984; 3,845,770; 3,916,899; 3,923,426; 3,987,790; 3,995,631; 4,016,880; 4,036,228; 4,111,202; 4,111,203; 4,203,440; 4,203,442; 4,210,139; 4,327,725; 4,360,019; 4,487,603; 4,627,850; 4,692,147; 4,725,852; 4,865,845; 4,911,616; 5,057,318; 5,059,423; 5,085,562; 5,112,614; 5,137,727; 5,219,278; 5,224,843; 5,234,692; 5,234,693; 5,271,724; 5,277,556; 5,728,396; 5,759,014; 5,759,015; 6,368,315; 6,464,687; 2004/0082908 and the like. All of these listed patents are incorporated herein by reference in their respective entireties.

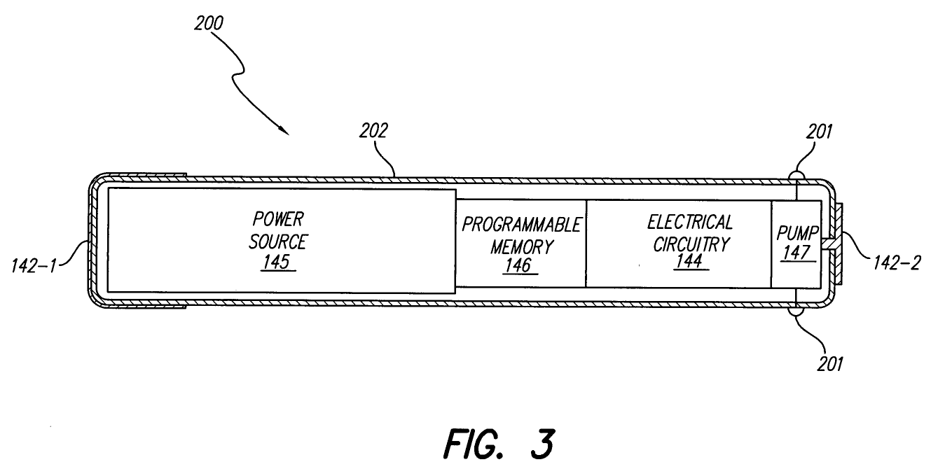

FIG. 3 illustrates an exemplary BION microstimulator (200) that may be used as the SCU (140; FIG. 2) described herein. Other configurations of the microstimulator (200) are possible, as shown in the above-referenced patents and as described further below.

As shown in FIG. 3, the microstimulator (200) may include the power source (145), the programmable memory (146), the electrical circuitry (144), and the pump (147) described in connection with FIG. 2. These components are housed within a capsule (202). The capsule (202) may be a thin, elongated cylinder or any other shape as best serves a particular application. The shape of the capsule (202) may be determined by the structure of the desired target, the surrounding area, and the method of implementation. For example, the diameter of the capsule (202) may be less than 5 millimeters (mm) and the length of the capsule (202) may be less than 40 mm in some instances. It will be recognized that the diameter, width, and/or length of the capsule (202) may be any size.

In some embodiments, the microstimulator (200) may include two leadless electrodes (142). Either or both of the electrodes (142) may alternatively be located at the ends of short, flexible leads as described in U.S. patent application Ser. No. 09/624,130, filed Jul. 24, 2000, which is incorporated herein by reference in its entirety. The use of such leads permits, among other things, electrical stimulation to be directed more locally to targeted tissue(s) a short distance from the surgical fixation of the bulk of the microstimulator (200), while allowing most elements of the microstimulator (200) to be located in a more surgically convenient site. This minimizes the distance traversed and the surgical planes crossed by the microstimulator (200) and any lead(s).

The external surfaces of the microstimulator (200) may advantageously be composed of biocompatible materials. For example, the capsule (202) may be made of glass, ceramic, metal, or any other material that provides a hermetic package that will exclude water vapor but permit passage of electromagnetic fields used to transmit data and/or power. The electrodes (142) may be made of a noble or refractory metal or compound, such as platinum, iridium, tantalum, titanium, titanium nitride, niobium or alloys of any of these, in order to avoid corrosion or electrolysis which could damage the surrounding tissues and the device.

As shown in FIG. 3, the microstimulator (200) may include one or more infusion outlets (201). The infusion outlets (201) facilitate the infusion of one or more drugs into a treatment site to treat chronic pain. The stimulator (200) of FIG. 3 also includes electrodes (142-1 and 142-2) at either end of the capsule (202). One of the electrodes (142) may be designated as a stimulating electrode to be placed close to the treatment site and one of the electrodes (142) may be designated as an indifferent electrode used to complete a stimulation circuit.

The microstimulator (200) may be implanted within a patient with a surgical tool such as a hypodermic needle, bore needle, or any other tool specially designed for the purpose. Alternatively, the microstimulator (200) may be implanted using endoscopic or laparoscopic techniques. In some embodiments, the microstimulator (200) is implanted adjacent to or near a target peripheral nerve causing chronic pain.

The leadless microstimulator (200) of FIG. 3 may alternatively be subcutaneously implanted just beneath the skin of the patient in a location where the patient feels pain. This location may be a considerable distance from the peripheral nerve causing the pain. For example, the peripheral nerve causing the pain may be located deep within the body of the patient. In these instances, the microstimulator (200) may be configured to stimulate neural tissue in the local vicinity of the implanted microstimulator (200). Stimulation of the neural tissue in the local vicinity of the implanted microstimulator (200) may effectively stimulate the peripheral nerve causing the pain because the neural tissue close to the surface of the skin is connected to the peripheral nerve via a number of small nerve fibers. Stimulation of the neural tissue in the local vicinity of the implanted microstimulator (200) is advantageous in many instances when the peripheral nerve causing the pain is located deep within the body of the patient and where invasive surgical procedures are required to implant the microstimulator (200) deep within the patient. Furthermore, the use of a leadless microstimulator (200) obviates the need to place a lead having one or more electrodes adjacent to or near the target peripheral nerve.

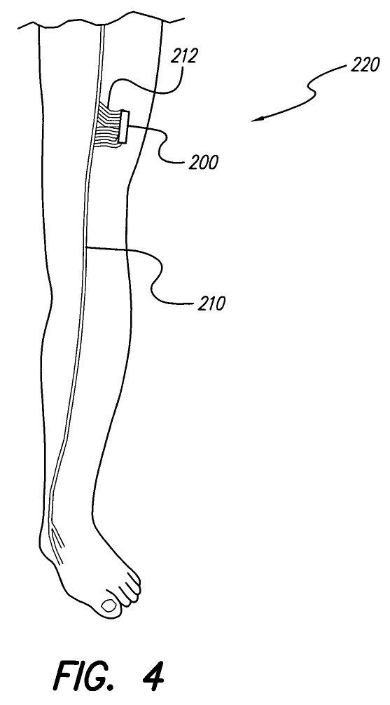

For example, as shown in FIG. 4, a leadless microstimulator (200) may be subcutaneously implanted within the leg (220) of a patient in a location where the patient feels pain. For illustrative purposes, it is assumed in the example of FIG. 4 that the patient feels pain in an upper thigh area (221) of the leg. The peripheral nerve causing the pain may be the tibial nerve (210). As shown in FIG. 4, the tibial nerve (210) may be located deep within the leg of the patient at the location (221) where the patient feels pain. Surgical procedures required to access the deeply located portion of the tibial nerve (210) may be dangerous and costly to the patient. However, neural tissue (212) located near the implanted microstimulator (200) is connected to the tibial nerve (210) via a network of nerve fibers and tissue. Hence, electrical and/or drug stimulation applied to the neural tissue (212) by the subcutaneously implanted microstimulator (200) may effectively stimulate the tibial nerve (210) and treat the pain felt by the patient.

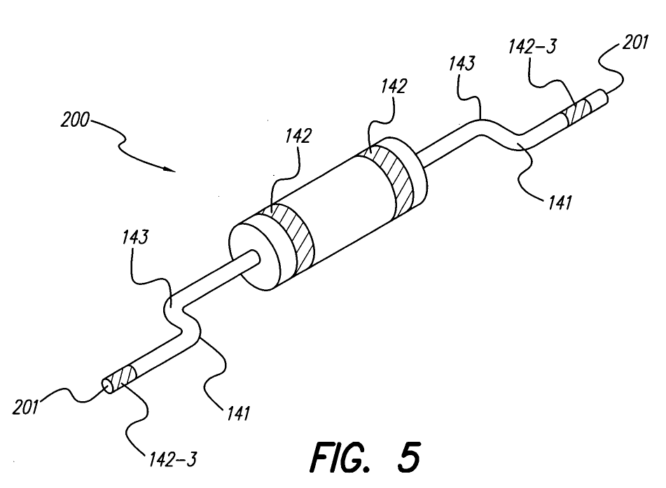

FIG. 5 shows that one or more catheters (143) may be coupled to the microstimulator (200). Infusion outlets (201) may be located at the end of a catheter (143) to facilitate drug infusion. As shown in FIG. 5, the catheters (143) may also serve as leads (141) having one or more electrodes (142-3). Thus, the catheters (143) and leads (141) of FIG. 5 permit infused drugs and/or electrical stimulation to be directed to a treatment site while allowing most elements of the microstimulator (200) to be located in a surgically convenient site.

Returning to FIG. 2, the SCU (140) may be configured to operate independently. Alternatively, the SCU (140) may be configured to operate in a coordinated manner with one or more additional SCUs (140), other implanted devices, or other devices external to the patient's body. For instance, a first SCU (140) may control or operate under the control of a second SCU (140), other implanted device, or other device external to the patient's body. The SCU (140) may be configured to communicate with other implanted SCUs (140), other implanted devices, or other devices external to the patient's body via an RF link, an untrasonic link, an optical link, or any other type of communication link. For example, the SCU (140) may be configured to communicate with an external remote control that is capable of sending commands and/or data to the SCU (140) and that is configured to receive commands and/or data from the SCU (140).

In order to determine the amount and/or type(s) of stimulating drug(s) and/or the strength and/or duration of electrical stimulation required to most effectively treat chronic pain, various conditions or indicators of chronic pain and/or a patient's response to treatment may be sensed or measured. These conditions include, but are not limited to, an amount of neural activity within a patient, amount of substance P in a peripheral nerve, amount of ENG in a peripheral nerve, neurotransmitter levels, hormone levels, and/or medication levels. In some embodiments, the SCU (140) may be configured to change the stimulation parameters in a closed loop manner in response to these measurements. The SCU (140) may be configured to perform the measurements. Alternatively, other sensing devices may be configured to perform the measurements and transmit the measured values to the SCU (140).

Thus, it is seen that one or more external appliances may be provided to interact with the SCU (140), and may be used to accomplish at least one or more of the following functions:

Function 1: If necessary, transmit electrical power to the SCU (140) in order to power the SCU (140) and/or recharge the power source (145).

Function 2: Transmit data to the SCU (140) in order to change the stimulation parameters used by the SCU (140).

Function 3: Receive data indicating the state of the SCU (140) (e.g., battery level, drug level, stimulation parameters, etc.).

Additional functions may include adjusting the stimulation parameters based on information sensed by the SCU (140) or by other sensing devices.

By way of example, an exemplary method of treating chronic pain within a patient may be carried out according to the following sequence of procedures. The steps listed below may be modified, reordered, and/or added to as best serves a particular application.

1. An SCU (140) is implanted so that its electrodes (142) and/or infusion outlet (149) are located in or on or near a peripheral nerve.

2. The SCU (140) is programmed to apply at least one stimulus to the peripheral nerve. The stimulus may include electrical stimulation and/or drug stimulation.

3. When the patient desires to invoke electrical and/or drug stimulation, the patient sends a command to the SCU (140) (e.g., via a remote control) such that the SCU (140) delivers the prescribed electrical and/or drug stimulation. The SCU (140) may be alternatively or additionally configured to automatically apply the electrical and/or drug stimulation in response to sensed indicators of chronic pain.

4. To cease electrical and/or drug stimulation, the patient may turn off the SCU (140) (e.g., via a remote control).

5. Periodically, the power source (145) of the SCU (140) is recharged, if necessary, in accordance with Function 1 described above.

For the treatment of any of the various types of chronic pain, it may be desirable to modify or adjust the algorithmic functions performed by the implanted and/or external components, as well as the surgical approaches. For example, in some situations, it may be desirable to employ more than one SCU (140), each of which could be separately controlled by means of a digital address. Multiple channels and/or multiple patterns of electrical and/or drug stimulation may thereby be used to deal with bilateral, complex, or multidimensional chronic pain.

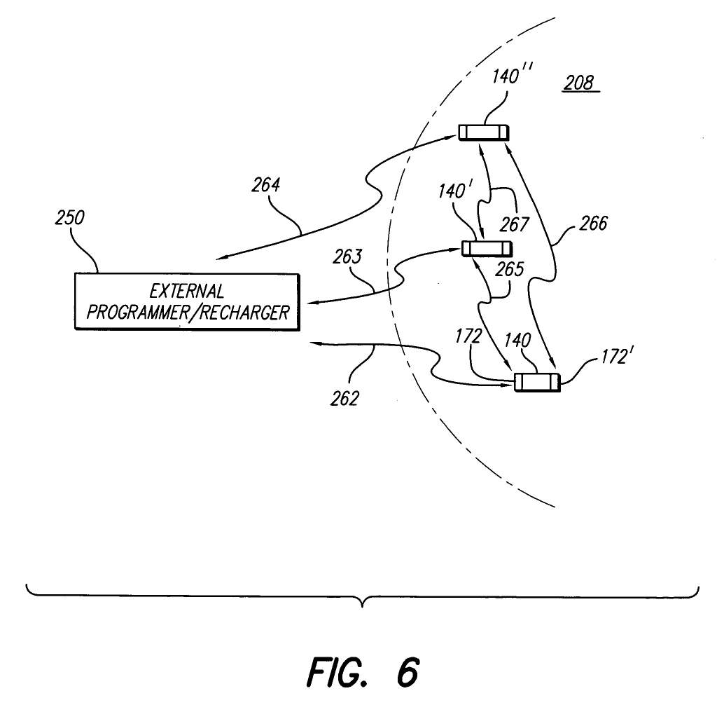

For instance, as shown in the example of FIG. 6, a first SCU (140) implanted beneath the skin of the patient (208) provides a first medication or substance; a second SCU (140′) provides a second medication or substance; and a third SCU (140″) provides electrical stimulation via electrodes (142, 142′). As mentioned earlier, the implanted devices may operate independently or may operate in a coordinated manner with other similar implanted devices, other implanted devices, or other devices external to the patient's body, as shown by the control lines (262-267) in FIG. 6. That is, an external controller (250) may be configured to control the operation of each of the implanted devices (140, 140′, and 140″). In some embodiments, an implanted device, e.g. SCU (140), may control or operate under the control of another implanted device(s), e.g. SCU (140′) and/or SCU (140″).

The preceding description has been presented only to illustrate and describe embodiments of the invention. It is not intended to be exhaustive or to limit the invention to any precise form disclosed. Many modifications and variations are possible in light of the above teaching.

1. A method of treating chronic pain within a patient, said method comprising:
subcutaneously implanting a system control unit substantially at or near a location where said patient feels said chronic pain; and
applying at least one stimulus to a peripheral nerve within said patient with an implanted system control unit in accordance with one or more stimulation parameters;
wherein said stimulus is configured to treat said chronic pain.

2. The method of claim 1, wherein said system control unit is coupled to one or more electrodes, and wherein said stimulus comprises a stimulation current delivered via said electrodes.

3. The method of claim 2, wherein said stimulation parameters control at least one or more of a frequency of said stimulation current, a pulse width of said stimulation current, a duty cycle of said stimulation current, a burst pattern of said stimulation current, and an amplitude of said stimulation current.

4. The method of claim 1, further comprising sensing at least one condition related to said chronic pain and using said at least one sensed condition to adjust one or more of said stimulation parameters.

5. The method of claim 1, wherein said system control unit comprises a micro stimulator.

6. The method of claim 1, wherein said system control unit comprises a leadless system control unit.

7. The method of claim 1, wherein said peripheral nerve comprises an occipital nerve.

8. A method of treating chronic pain within a patient, said method comprising:
applying at least one stimulus to a peripheral nerve within said patient with an implanted system control unit in accordance with one or more stimulation parameters;
wherein said stimulus comprises stimulation via one or more drugs delivered to said peripheral nerve and wherein said stimulus is configured to treat said chronic pain.

9. The method of claim 8, wherein said system control unit is coupled to one or more electrodes, and wherein said stimulus further comprises a stimulation current delivered via said electrodes.

10. The method of claim 8, further comprising subcutaneously implanting said system control unit substantially at or near a location where said patient feels said chronic pain.

11. The method of claim 8, wherein said stimulation parameters control at least one or more of an amount of said one or more drugs delivered to said peripheral nerve and a rate of delivery of said one or more drugs to said peripheral nerve.

12. The method of claim 8, further comprising sensing at least one condition related to said chronic pain and using said at least one sensed condition to adjust one or more of said stimulation parameters.

13. The method of claim 8, wherein said system control unit comprises a micro stimulator.

14. The method of claim 8, wherein said system control unit comprises a leadless system control unit.

15. The method of claim 8, wherein said peripheral nerve comprises an occipital nerve.

16. A system for treating chronic pain within a patient, said system comprising:
a system control unit configured to apply a stimulus to a peripheral nerve within said patient in accordance with one or more stimulation parameters;
wherein said system control unit configured to be subcutaneously implanted substantially at or near a location where said patient feels said chronic pain and wherein said stimulus is configured to treat said chronic pain.

17. The system of claim 16, further comprising a pump for delivering one or more drugs, wherein said stimulus comprises stimulation via said one or more drugs delivered by said pump to said peripheral nerve.

18. The system of claim 17, wherein said stimulation parameters control at least one or more of an amount of said one or more drugs delivered to said peripheral nerve and a rate of delivery of said one or more drugs to said peripheral nerve.

19. The system of claim 16, further comprising:
one or more electrodes coupled to said system control unit; and
a pump for delivering one or more drugs;
wherein said stimulus comprises a stimulation current delivered by said system control unit via said electrodes and stimulation via said one or more drugs delivered by said pump.

20. The system of claim 16, further comprising:
a sensor device for sensing at least one condition;
wherein said system control unit uses said at least one sensed condition to adjust one or more of said stimulation parameters.

21. The system of claim 16, wherein said system control unit comprises a micro stimulator.

22. The system of claim 16, wherein said system control unit comprises a leadless system control unit.

23. The system of claim 16, wherein said peripheral nerve comprises an occipital nerve.

24. A system for treating chronic pain within a patient, said system comprising:
means for subcutaneously implanting a system control unit substantially at or near a location where said patient feels said chronic pain; and
means for applying at least one stimulus to a peripheral nerve within said patient with an implanted system control unit in accordance with one or more stimulation parameters;
wherein said stimulus is configured to treat said chronic pain.

25. The system of claim 24, wherein said system control unit is coupled to one or more electrodes, and wherein said stimulus comprises a stimulation current delivered via said electrodes.

26. The system of claim 24, wherein said stimulus comprises stimulation via one or more drugs delivered to said peripheral nerve.

27. A system for treating chronic pain within a patient, said system comprising:
means for applying a drug stimulation to a peripheral nerve within said patient with an implanted system control unit in accordance with one or more stimulation parameters;
wherein said drug stimulation is configured to treat said chronic pain.

28. The system of claim 27, further comprising means for applying an electrical stimulation to said peripheral nerve within said patient with said implanted system control unit in accordance with said one or more stimulation parameters.
